(19) United States
(12) Patent Application Publication
(10) Pub. No.: US 2005/0137182 A1
(43) Pub. Date: Jun. 23, 2005

(54) Novel crystalline form of cefdinir

(76) Inventors: Ramesh Dandala, Hyderabad (IN); Meenakshisunderam Sivakumaran, Hyderabad (IN)

Correspondence Address: WINSTON & STRAWN LLP, 1700 K STREET, N.W., WASHINGTON, DC, 20006, US

(21) Appl. No.: 10/976,230

(22) Filed: Oct. 29, 2004

Related U.S. Application Data
(63) Continuation-in-part of application No. 10/634,978, filed on Feb. 24, 2004.

(30) Foreign Application Priority Data
Jun. 2, 2003 (IN) 440/MAS/2003

(51) Int. Cl. A61K 31/545; C07D 501/14

(52) U.S. Cl. 514/202; 540/222

(57) ABSTRACT

The present invention relates to novel crystalline form of Cefdinir, 7β-[(Z)-2-(2-amino-4-thiazolyl)-2-hydroxyiminoacetamido]-3-vinyl-3-cephem-4-carboxylic acid, herein referred as cefdinir crystal B, processes for preparing cefdinir crystal B, and the incorporation of cefdinir crystal B in pharmaceutical compositions.

### CROSS REFERENCE TO RELATED APPLICATIONS

This application is a continuation-in-part of U.S. application Ser. No. 10/634,978, filed Aug. 4, 2003, the entire content of which is expressly incorporated herein by reference thereto.

### FIELD OF INVENTION

The present invention relates to a new inventive polymorph of crystalline cefdinir of Formula I, processes of making the same, and pharmaceutical compositions containing the new crystalline cefdinir of Formula I.

### BACKGROUND OF THE INVENTION

Cefdinir of Formula I is a very useful antimicrobial agent and is chemically known as 7β-[(Z)-2-(2-amino-4-thiazolyl)-2-hydroxyiminoacetamido]-3-vinyl-3 -cephem-4-carboxylic acid.

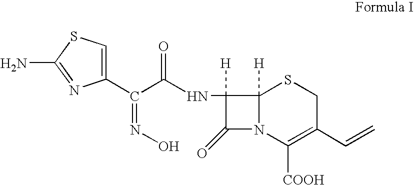

Cefdinir is an oral antibiotic and was first disclosed in U.S. Pat. No. 4,559,334. The product obtained according to above invention was crystalline like amorphous product, not a crystalline product.

U.S. Pat. No. 4,935,507 discloses methods of producing crystalline cefdinir that offered better filtration rate, high purity and stable cefdinir suitable for pharmaceutical preparation. This material was prepared by treating amorphous cefdinir with sodium bicarbonate solution and the resulting aqueous solution was subjected to column chromatography and then adjusting the pH between 1-2 at 35-40° C. followed by cooling to obtain cefdinir crystal A. Alternatively, amorphous cefdinir was dissolved in methanol and to this solution added water at 35° C., stirred and allowed to stand at room temperature to obtain cefdinir crystal A. Cefdinir crystal A as disclosed in U.S. Pat. No. 4,935,507 has a water content of about 0.8% (measured by Karl Fisher method).

Although U.S. Pat. No. 4,935,507 claims crystalline form A, advantages have yet to be realized by other, heretofore undiscovered, forms of cefdinir. These are now provided by the present invention.

### SUMMARY OF THE INVENTION

The present invention includes a novel crystalline form of cefdinir. Polymorphism is the property of some molecules and molecular complexes to assume more than one crystalline or amorphous form in solid state. A molecule like cefdinir of Formula I may give to a variety of solids having distinct physical properties like solubility, melting point, powder X-ray diffractogram (XRD). The differences in the physical properties of polymorphs result from the orientation and intermolecular interactions of adjacent molecules (complexes) in the bulk solid. Accordingly, polymorphs are distinct solids sharing the same molecular formula, which may be thought of as analogous to unit cell metallurgy, yet having distinct advantageous and or disadvantageous physical properties compared to other forms in the polymorph family.

The present invention provides a new crystalline form of cefdinir having moisture content in the range of about 5.5-7%, but typically 6-7%, (determined by the Karl Fisher (KF) method) amounting closely to sesquihydrate and is herein referred to as cefdinir crystal B. Further, the major advantages realized in the present invention are avoiding the use of chromatographic technique and highly pure cefdinir crystal B is obtained. The present invention provides a stable, nontoxic, non-solvate crystalline modification of cefdinir, which is substantially free of impurities.

The present invention specifically relates to a crystalline 7β-[(Z)-2-(2-amino-4-thiazolyl)-2-hydroxyiminoacetamido]-3-vinyl-3-cephem-4-carboxylic acid (cefdinir crystal B), the X-ray diffraction spectrum (using copper cathode) of which has the following characteristics:

| | Interplanar | Relative |
|---|---|---|
| Diffraction Angle | Spacing (d) | Intensity |
| (2θ°) | (Å) | (%) |
| 5.8 | 15.16 | 24 |
| 7.8 | 11.38 | 30 |
| 8.0 | 11.02 | 30 |
| 11.7 | 7.55 | 100 |
| 15.6 | 5.68 | 41 |
| 16.1 | 5.50 | 61 |
| 18.6 | 4.77 | 43 |
| 19.4 | 4.58 | 24 |
| 20.9 | 4.24 | 33 |
| 21.2 | 4.19 | 43 |
| 22.3 | 3.99 | 60 |
| 22.5 | 3.95 | 25 |
| 24.4 | 3.64 | 43 |
| 25.6 | 3.47 | 37 |
| 26.2 | 3.40 | 41 |

Thus, the present cefdinir crystal B shows X-ray diffraction peaks at the diffraction angles of about 5.8±0.2, 11.7±0.2, 16.1±0.2, 18.6±0.2, 21.2±0.2, 22.3±0.2, 24.4±0.2 and 26.2±0.2 two theta degrees, which correspond to the interplanar spacing values of about 15.16, 7.55, 5.50, 4.77, 4.19, 3.99, 3.64, and 3.40 Å. The present cefdinir crystal B can also be characterized by infrared absorption spectrum pattern having characteristic peaks at approximately 1017, 1049, 1121, 1134, 1191, 1428, 1545, 1613, 1667, 1780, 3295 and 3595 cm⁻¹.

Further, the present cefdinir crystal B has a water content of about 5.5 to 7% by weight. The present crystalline cefdinir compound has increased storage stability, relative to the cefdinir crystal A which has a water content less than 5.5% by weight, at a storage temperature of about 40° C. and relative humidity of about 75%. Advantageously, only about 0.5% or less of the present cefdinir crystal B compound degrades over a period of at least 3 months under accelerated storage conditions.

The present invention also relates to a pharmaceutical composition comprising a therapeutically effective amount of cefdinir crystal B and a pharmaceutically acceptable carrier. In addition, the invention relates to various processes for preparing the present cefdinir crystal B.

### BRIEF DESCRIPTION OF ACCOMPANYING DRAWINGS

FIG. 1 is a characteristic X-ray powder diffractogram of cefdinir crystal B, with
Vertical axis: Intensity (CPS)
Horizontal axis: Two theta (degrees).

FIG. 2 is a X-ray powder diffractogram of cefdinir crystal A, with
Vertical axis: Intensity (CPS)
Horizontal axis: Two theta (degrees).

FIG. 3 is the infrared absorption spectrum of cefdinir crystal B.

FIG. 4 is the infrared absorption spectrum of cefdinir crystal A.

### DETAILED DESCRIPTION OF THE PREFERRED EMBODIMENTS

The present invention relates to a novel crystalline form of cefdinir (hereinafter “cefdinir crystal B”). More particularly, the novel form may contain water up to about 5.5 to 7% by weight but typically close to 6.3% w/w which corresponds to the stoichiometric value of about 1.5 mole of water per mole of cefdinir. The novel crystal B of cefdinir of the present invention may be characterized by powder X-ray diffraction and infrared absorption spectrum. Thus powder X-ray diffraction of novel cefdinir crystal B and crystal A were determined on Seifert XRD 3003TT system using a copper target X-ray tube, a nickel filter and the sample was placed in a pyrex glass holder. The scan rate was 1.8 degrees, two theta per minute, with the step size of 0.03 degrees two theta over the range from 5 to 50 degrees.

The novel cefdinir crystal B compound has powder X-ray diffraction pattern essentially as shown in FIG. 1 and Table 1, and the powder X-ray diffraction pattern reported for crystal A is given in FIG. 2 and Table 2. The powder X-ray diffraction patterns are expressed in terms of the diffraction angle (2θ) and relative intensities. Table 1 also shows the interplanar spacing (d) values of the X-ray diffraction spectrum that correspond to the diffraction angle values.

The diffraction angle (2θ) and interplanar spacing (d) parameters of an X-ray diffraction spectrum are related by the following Bragg's equation: nλ=2d Sin θ, where n=1;
λ=wavelength of the cathode used;
d=interplanar spacing; and
θ=diffraction angle.

The wavelength of the cathode used (λ of copper Kα) is 1.54 Å. Thus, d=nλ/2 Sin θ=1.54/(2 Sin θ).

TABLE 1
Significant Diffraction Angle (2θ), Interplanar Spacing (d)
and Relative Intensities for Cefdinir Crystal B

| 2θ | d | Relative Intensity (%) |
|---|---|---|
| 5.8 | 15.16 | 24 |
| 7.8 | 11.38 | 30 |
| 8.0 | 11.02 | 30 |
| 11.7 | 7.55 | 100 |
| 15.6 | 5.68 | 41 |
| 16.1 | 5.50 | 61 |
| 18.6 | 4.77 | 43 |
| 19.4 | 4.58 | 24 |
| 20.9 | 4.24 | 33 |
| 21.2 | 4.19 | 43 |
| 22.3 | 3.99 | 60 |
| 22.5 | 3.95 | 25 |
| 24.4 | 3.64 | 43 |
| 25.6 | 3.47 | 37 |
| 26.2 | 3.40 | 41 |

As FIG. 1 and Table 1 show, some of the highest and most significant peaks occur at the diffraction angles (2θ) of about: 5.8±0.2, 11.7±0.2, 16.1±0.2, 18.6±0.2, 21.2±0.2, 22.3±0.2, 24.4±0.2 and 26.2±0.2 degrees. These 2θ values correspond, respectively, to interplanar spacing (d) of about: 15.16, 7.55, 5.50, 4.77, 4.19, 3.99, 3.64 and 3.40 Å. It is believed that the peaks represented in Table 1 are primarily responsible for the properties of the compound.

In contrast, as shown in FIG. 2 and Table 2, the X-ray diffraction spectrum of cefdinir crystal A shows significant peaks at the diffraction angle values (2θ) of, for example: about 11.7±0.2, 14.7±0.2, 17.8±0.2, 21.5±0.2, 21.9±0.2, 23.4±0.2, 24.5±0.2, and 25.4±0.2 degrees. Importantly, the highest X-ray diffraction peak occurs at the diffraction angle (2θ) of about 21.5 degrees for cefdinir crystal A, while that for cefdinir crystal B occurs at the diffraction angle (2θ) of about 11.7 degrees (corresponding to the interplanar spacing of about 7.55 Å).

TABLE 2
Significant Diffraction Angle (2θ) and Relative
Intensities for Cefdinir Crystal A

| 2θ | Relative Intensity (%) |
|---|---|
| 6.9 | 9 |
| 8.8 | 8 |
| 11.7 | 21 |
| 12.5 | 19 |
| 14.7 | 89 |
| 16.5 | 20 |
| 17.8 | 59 |
| 18.8 | 22 |
| 21.5 | 100 |
| 21.9 | 73 |
| 23.4 | 42 |
| 24.5 | 87 |
| 25.4 | 20 |

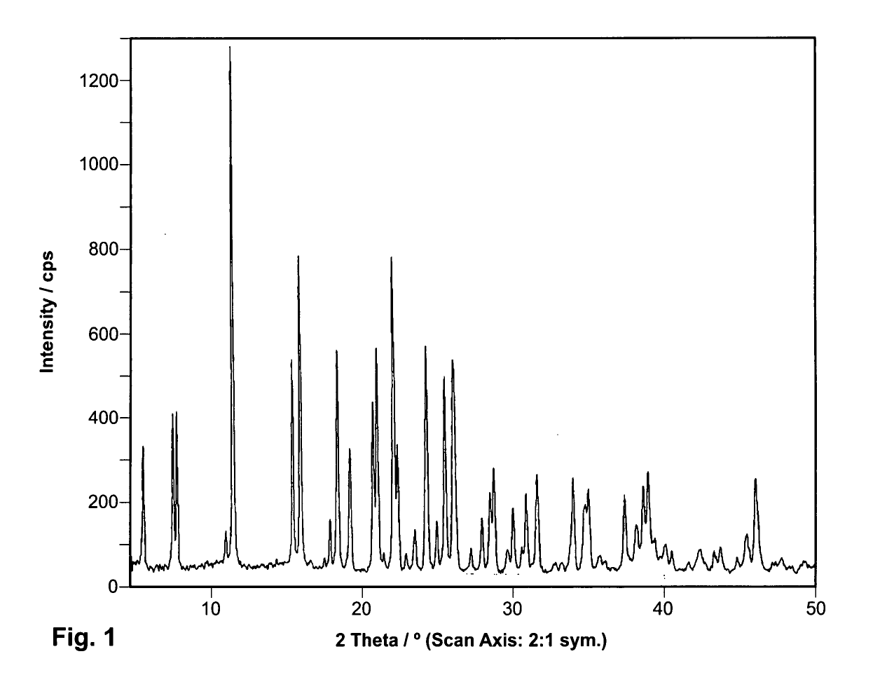

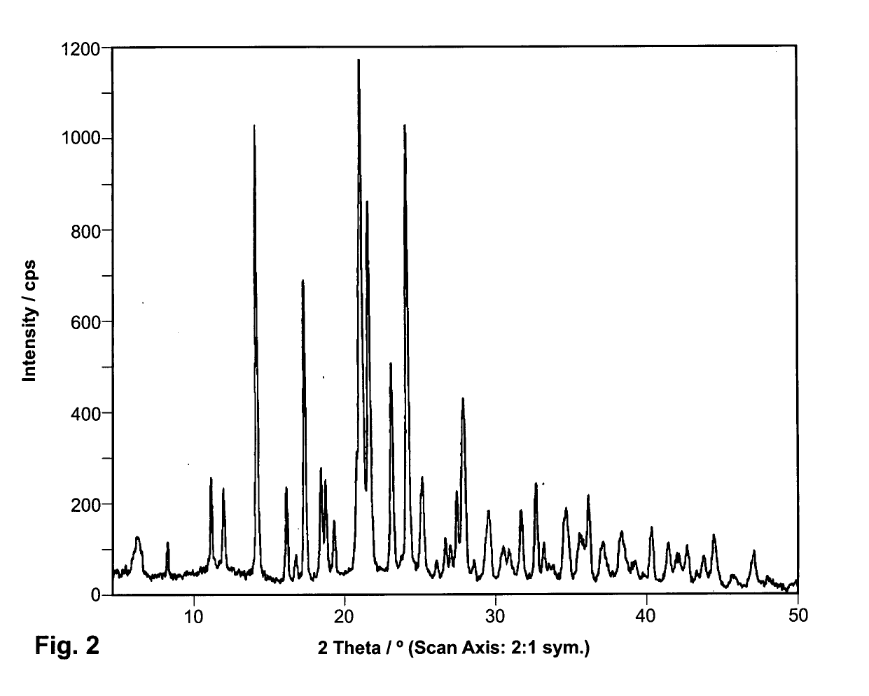

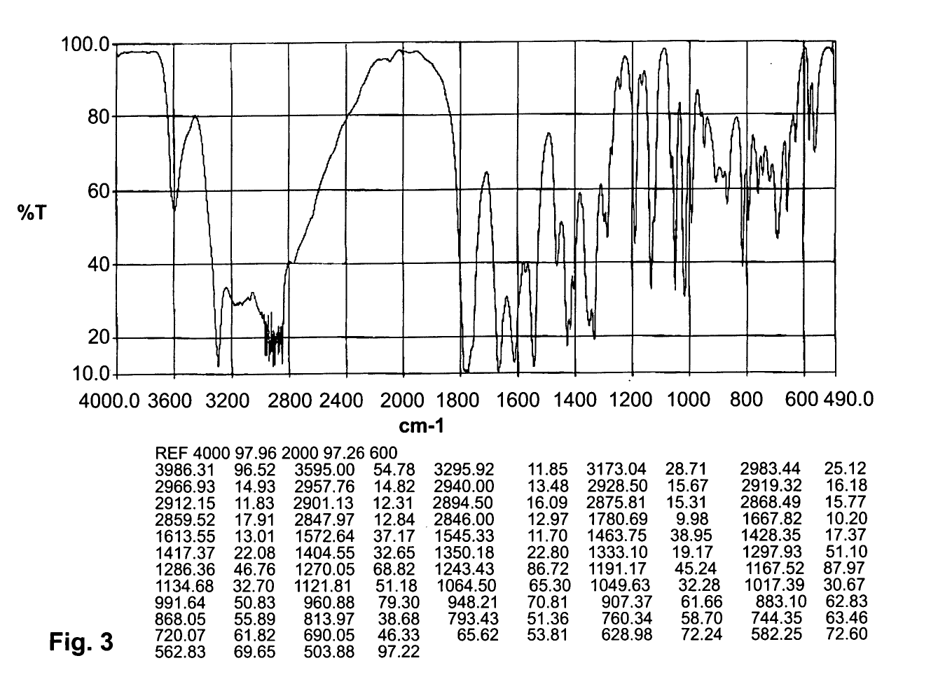

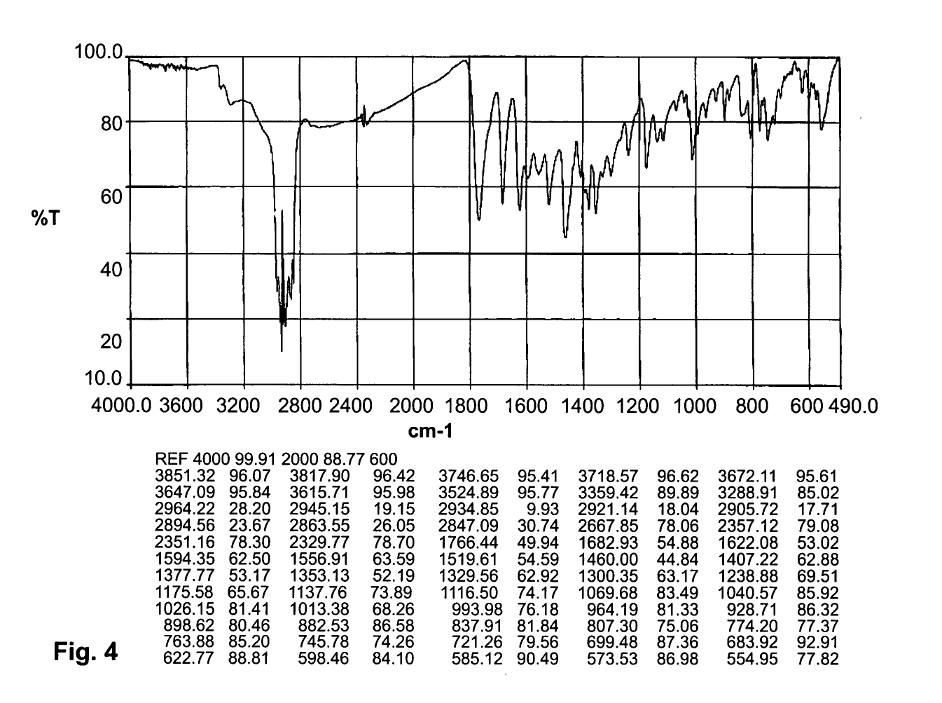

FIGS. 3 and 4 are representative infrared absorption spectra of cefdinir crystal B and cefdinir crystal A, which were determined on Perkin Elmer-spectrum ONE infrared spectrophotometer.

The infrared absorption spectrum of cefdinir crystal B (FIG. 3) shows characteristics peaks at approximately 1017, 1049, 1121, 1134, 1191, 1428, 1545, 1613, 1667, 1780, 3295 and 3595 cm⁻¹, whereas the infrared spectrum of cefdinir crystal A (FIG. 4) shows characteristics peaks at about 1013, 1175, 1460, 1519, 1556, 1594, 1622, 1682 and 1766 cm⁻¹.

It is evident from the above data that the novel crystalline form of present invention is different from the cefdinir crystal A reported in U.S. Pat. No. 4,935,507. The powder X-ray diffraction pattern of crystal B shows maximum peak at about 11.7±0.2 degree two theta whereas crystal A shows maximum peak at about 21.5±0.2 degree two theta.

Furthermore, Applicants have found that this novel cefdinir crystal B has excellent storage stability characteristics and is suitable for pharmaceutical formulations.

Stress stability studies of cefdinir crystal B were performed by keeping samples of cefdinir crystal B under the conditions of 60° C.±2° C. for 15 days, and the purity and assay of the samples were determined before and after the stress test by high performance liquid chromatography. Remarkably, the studies showed that cefdinir crystal B did not degrade even after keeping the samples at 60° C.±2° C. for 15 days, thus indicating a good stability for a cephalosporin compound. Table 3 summaries the results of the stress test studies.

TABLE 3
Stress Stability Data for Cefdinir Crystal B

| | | Assay |
|---|---|---|
| | Purity | (by HPLC on anhydrous |
| Sample | (by HPLC, % change) | basis, % change) |
| 1 | −0.21% | 0% |
| 2 | −0.33% | 0% |

Furthermore, a comparison of the stability data of cefdinir crystal A with that of cefdinir crystal B shows that, while cefdinir crystal A is as stable as crystal B under the normal storage conditions (at 25° C.±2° C./60% RH±5% RH) as indicated by the real time storage data at 3 months station in Table 4, cefdinir crystal B is found to have a superior stability under accelerated storage conditions as compared to crystal A. Table 4 shows that the initial purity of cefdinir crystal B is comparatively much higher than that of crystal A. In addition, during accelerated storage up to 3 months (at 40° C.±2° C./75% RH±5% RH), cefdinir crystal B does not show any appreciable drop in assay or purity as compared to that observed in crystal A material. For example, cefdinir crystal B shows a change in the assay of much less than 0.5% and typically only about 0.1 to 0.2% after 3 months under accelerated storage conditions, whereas cefdinir crystal A shows a change in the assay of about 1.8% after 3 months.

TABLE 4
Comparison of Stability: Cefdinir Crystal B vs. Cefdinir Crystal A

| | | Accelerated | | | | Real Time | |
|---|---|---|---|---|---|---|---|
| | | Storage conditions: | | | | Storage conditions: | |
| | | 40° C. ± 2° C./ | | | | 25° C. ± 2° C./ | |
| | | 75% RH ± 5% RH | | | | 60% RH ± 5% RH | |
| | PARAMETER | Initial | 1 Month | 2 Months | 3 Months | Initial | 3 Months |
| Crystal A | Assay (% w/w, | 96.3 | −0.5 | −1.6 | −1.8 | 96.3 | −0.2 |
| | by HPLC OAB), % change | | | | | | |
| | Total RS (% area | 0.82 | 1.98 | 2.25 | 2.06 | 0.82 | 1.18 |
| | normalization, by HPLC) | | | | | | |
| Crystal B | Assay (% w/w, | 99.9 | 0.0 | 0.0 | −0.1 | 99.9 | 0.0 |
| Sample 1 | by HPLC OAB), % change | | | | | | |
| | Total RS (% area | 0.64 | 0.72 | 1.10 | 0.90 | 0.64 | 0.53 |
| | normalization, by HPLC) | | | | | | |
| Crystal B | Assay (% w/w, | 99.4 | — | 0.0 | −0.2 | 99.4 | 0.0 |
| Sample 2 | by HPLC OAB), % change | | | | | | |
| | Total RS (% area | 0.59 | 0.70 | 0.92 | 1.08 | 0.59 | 0.61 |
| | normalization, by HPLC) | | | | | | |

RS = Related substances
OAB = On Anhydrous Basis
RH = Relative Humidity

The present invention also relates to the process for preparing the novel cefdinir crystal B.

According to one embodiment, the process comprises the step of condensation of 7-amino-3-vinyl-3-cephem-4-carboxylate 4-methoxybenzyl ester hydrochloride with 2-benzothiazolyl (Z)-2-(2-amino-4-thiazolyl)-2-trityloxyiminothioacetate in the presence of trialkylamine in any suitable solvent such as N,N-dimethylacetamide, N,N-dimethylformamide, methylene dichloride and like and mixture thereof. Specifically the condensation is carried out at a temperature range of 40-50° C. After completion of reaction, the reaction mass is cooled and product is extracted into suitable organic solvent and washed with dilute base and water. The suitable solvent can be selected from methylene dichloride, chloroform, toluene, ethylene dichloride etc., most preferably methylene dichloride. Further, organic layer is treated with trifluoroacetic acid at a temperature of about 10-15° C. for 4-5 h to remove carboxyl protecting groups. Thereafter, the organic layer was cooled to 0° C. and water was added. The layers were separated, and on cooling the aqueous layer, trifluoroacetic acid salt of cefdinir precipitated out, which is isolated by filtration.

Further, the wet cefdinir trifluoroacetic acid salt is suspended in water and neutralized with aqueous ammonia at a temperature of 0-30° C., most preferably at 20-25° C., to obtain highly purified cefdinir crystal B.

A further aspect of the present invention is a process to produce cefdinir crystal B from cefdinir crystal A.

This process comprises of suspending cefdinir crystal A in water at 35-40° C. and treating with trifluoroacetic acid to prepare cefdinir.trifluoroacetic acid salt. The trifluoroacetic acid salt of cefdinir is isolated in high purity. Typically purity is analyzed by HPLC and is greater than 99.5% and generally closer to 99.6%. The trifluoroacetic acid salt of cefdinir is then neutralized by adjusting the pH to 3.0-3.2 with aqueous ammonia in water at 0-30° C. preferably at 20-25° C. to obtain highly pure cefdinir crystal B as off white solid. Typically, the purities are greater than 99% by HPLC in commercial lots. The trifluoroacetic acid salt of cefdinir is preferably used as wet material without drying.

The cefdinir crystal A can be prepared by methods known in the art (U.S. Pat. No. 4,935,507) and then can easily be converted to cefdinir crystal B.

The present invention therefore provides a novel crystalline form, crystal B of cefdinir and a method of its preparation, which is amenable to large-scale production, and suitable for formulation. The novel crystalline form, crystal B of cefdinir of the present invention is readily filterable and easily dried. Moreover, the cefdinir crystal B prepared is of high purity of typically greater than 99.5% as seen by HPLC. The novel crystalline form shows excellent storage stability and hence suitable for formulation. The novel crystal B of cefdinir may contain 5.5-7% of water and has a decomposition range of 188-192° C.

Having thus described the various aspects of the present invention, the following examples are provided to illustrate specific embodiments of the present invention. They are not intended to be limiting in anyway.

### EXAMPLES

### Example 1

### Preparation of 7β-[(Z)-2-(2-Amino-4-thiazolyl)-2-hydroxyiminoacetamido-3-vinyl-3-cephem-4-carboxylic acid.trifluoroacetic acid salt (Cefdinir.Trifluoroacetic Acid Salt)

50 g of 7-Amino-3-vinyl-3-cephem-4-carboxylate-4-methoxybenzyl ester hydrochloride (0.130 mol) and 71.5 g of 2-benzothiazolyl (Z)-2-(2-amino-4-thiazolyl)-2-trityloxyiminothioacetate (0.124 mol) were suspended in 250 ml of N,N-dimethylacetamide and 12.5 g of triethylamine (0.124 mol) was added thereto. Then the mixture was stirred for 3 hours at 40-50° C. After cooling to 10° C., methylene dichloride (750 ml) was added followed by demineralised water (1000 ml) and stirred for 10 minutes at the same temperature. Layers were separated and the organic layer was washed with dilute sodium hydroxide solution and water respectively. The methylene dichloride layer was cooled to 10° C. Trifluoroacetic acid (300 ml) was added over a period of 30 minutes at 10-15° C. and stirred for 4 hours at the same temperature. Thereafter, cooled the reaction solution to 0° C. Demineralised water (875 ml) was added and separated the layers. Aqueous extract was cooled to 0° C. and stirred for 60 minutes. The precipitate thus obtained was filtered and washed with ice-cooled water (125 ml) to obtain trifluoroacetic acid salt of cefdinir as an off white crystalline solid having purity 99.5% by HPLC.

¹H-NMR in DMSO-d₆: δ(ppm);3.58 and 3.83 (Abq, 2H, J=17.84), 5.20 (d, 1H, J=4.94 Hz),
5.32 (d, 1H, J=11.25 Hz), 5.60 (d, 1H, J=17.56 Hz),
5.79 (dd, 1H, J=4.94 Hz and 8.23 Hz), 6.76 (s, 1H),
6.92 (dd, 1H, J=11.25 Hz and 17.56 Hz),
9.60 (d, 1H, J=7.96 Hz)

### Example 2

### Preparation of 7β-[(Z)-2-(2-Amino-4-thiazolyl)-2-hydroxyiminoacetamido]-3-vinyl-3-cephem-4-carboxylic acid (Cefdinir Crystal B)

The wet product 7β-[(Z)-2-(2-amino-4-thiazolyl)-2-hydroxyiminoacetamido]-3-vinyl-3-cephem-4-carboxylic acid.trifluoroacetic acid salt obtained in Example-1 was suspended in water (875 ml) and cooled to 10° C. pH was adjusted to 3.0-3.2 with aqueous ammonia solution at 20-25° C. and filtered, washed with water (250 ml) and thus dried to obtain 24 g of cefdinir crystal B as a off white solid. (HPLC Purity: 99.6%).

Water content (% w/w, by KF): 7%; Melting point: 190° C. (decompose).

¹H-NMR in DMSO-d₆: δ(ppm);3.55 and 3.84 (Abq, 2H, J=17.56 Hz),
5.19 (d, 1H, J=4.94 Hz), 5.31 (d, 1H, J=11.25 Hz),
5.59 (d, 1H, J=17.56 Hz),
5.79 (dd, 1H, J=4.94 Hz and 8.23 Hz),
6.66 (s, 1H), 6.90 (dd, 1H, J=11.25 Hz and 17.56 Hz),
7.13 (bs, 2H), 9.48 (d, 1H, J=8.23 Hz).

### Example 3

### Preparation of 7β-[(Z)-2-(2-Amino-4-thiazolyl)-2-hydroxyiminoacetamido]-3-vinyl-3-cephem-4-carboxylic acid (Cefdinir Crystal B)

10 g of Cefdinir crystal A was suspended in 300 ml of demineralised water at 30-35° C. 30 ml of trifluoroacetic acid was added in 15 minutes at 30-35° C. to get a clear solution. After about 15-20 minutes, precipitation was started and stirred for 60 minutes at 40° C. Precipitate thus obtained, was filtered, washed with chilled water (20 ml) to obtain trifluoroacetic acid salt of cefdinir. This salt without further drying was suspended in demineralised water (200 ml) at 20-25° C. and the pH was adjusted to 3.0-3.2 with aqueous ammonia solution at 20-25° C. Stirred the resulting slurry for 30 minutes, filtered, washed with water (50 ml) and dried to get cefdinir crystal B as an off white solid. (HPLC Purity: 99.6%)

Water content (% w/w, by KF): 6.67%; Melting point: 190° C. (decompose)

¹H-NMR in DMSO-d₆: δ(ppm);3.56 and 3.84 (Abq, 2H, J=17.56 Hz), 5.19 (d, 1H, J=4.94 Hz),
5.31 (d, 1H, J=11.25 Hz), 5.59 (d, 1H, J=17.56 Hz),
5.79 (dd, 1H, J=4.94 Hz and 8.23 Hz), 6.67 (s, 1H),
6.91 (dd, 1H, J=11.25 Hz and 17.56 Hz), 7.15 (bs, 2H),
9.50 (d, 1H, J=8.23 Hz).

1. A crystalline 7β-[(Z)-2-(2-amino-4-thiazolyl)-2-hydroxyiminoacetamido]-3-vinyl-3-cephem-4-carboxylic acid (cefdinir crystal B), the X-ray diffraction spectrum of which shows peaks at the diffraction angles of about 5.8±0.2, 11.7±0.2, 16.1±0.2, 18.6±0.2, 21.2±0.2, 22.3±0.2, 24.4±0.2 and 26.2±0.2 two theta degrees.

2. Crystalline cefdinir of claim 1 which is characterized by infrared absorption spectrum pattern having characteristic peaks at approximately 1017, 1049, 1121, 1134, 1191, 1428, 1545, 1613, 1667, 1780, 3295 and 3595 cm⁻¹.

3. Crystalline cefdinir of claim 1, which contains water in the range of about 5.5 to 7% by weight.

4. A crystalline form of cefdinir, the X-ray diffraction spectrum of which shows peaks at the interplanar spacing (d) of about 15.16, 7.55, 5.50, 4.77, 4.19, 3.99, 3.64, and 3.40 Å.

5. Crystalline form of cefdinir according to claim 4, which is characterized by infrared absorption spectrum pattern having characteristic peaks at approximately 1017, 1049, 1121, 1134, 1191, 1428, 1545, 1613, 1667, 1780, 3295 and 3595 cm⁻¹.

6. Crystalline form of cefdinir according to claim 4, which contains water in the range of about 5.5 to 7% by weight.

7. Crystalline form of cefdinir according to claim 4, the X-ray spectrum of which has the following characteristics:

| | Interplanar | Relative |
|---|---|---|
| Diffraction Angle | Spacing (d) | Intensity |
| (2θ°) | (Å) | (%) |
| 5.8 | 15.16 | 24 |
| 7.8 | 11.38 | 30 |
| 8.0 | 11.02 | 30 |
| 11.7 | 7.55 | 100 |
| 15.6 | 5.68 | 41 |
| 16.1 | 5.50 | 61 |
| 18.6 | 4.77 | 43 |
| 19.4 | 4.58 | 24 |
| 20.9 | 4.24 | 33 |
| 21.2 | 4.19 | 43 |
| 22.3 | 3.99 | 60 |
| 22.5 | 3.95 | 25 |
| 24.4 | 3.64 | 43 |
| 25.6 | 3.47 | 37 |
| 26.2 | 3.40 | 41 |

8. A crystalline cefdinir compound having a water content greater than 5.5% by weight, which has increased storage stability, relative to a crystalline cefdinir having a water content less than 5.5% by weight, at a storage temperature of about 40° C. and relative humidity of about 75%.

9. Crystalline cefdinir compound according to claim 8, which contains water in the range of about 5.5 to 7% by weight.

10. Crystalline cefdinir compound according to claim 9, wherein 0.5% or less of the compound degrades over a period of at least 3 months under accelerated storage conditions.

11. A pharmaceutical composition comprising a therapeutically effective amount of cefdinir crystal B of claim 1 and a pharmaceutically acceptable carrier.

12. A pharmaceutical composition comprising a therapeutically effective amount of the crystalline form of cefdinir according to claim 8 and a pharmaceutically acceptable carrier.

13. A process for preparing crystalline 7β-[(Z)-2-(2-amino-4-thiazolyl)-2 hydroxyiminoacetamido]-3-vinyl-3-cephem-4-carboxylic acid (cefdinir crystal B) which comprises the steps of:
reacting crystals A of cefdinir in water with trifluoroacetic acid at about 35-40° C. to form cefdinir trifluoroacetic acid salt;
optionally isolating the cefdinir.trifluoroacetic acid salt;
neutralizing the cefdinir.trifluoroacetic acid salt by treatment with a base in water at a temperature between about 0° C. to 30° C.; and
isolating cefdinir crystal B by filtration.

14. The process according to claim 13, wherein the base used for neutralization is ammonia.

15. The process according to claim 13, wherein the neutralization step is conducted at a temperature range of about 0 to 30° C.

16. A process for preparing crystalline 7β-[(Z)-2-(2-amino-4-thiazolyl)-2 hydroxyiminoacetamido]-3-vinyl-3-cephem-4-carboxylic acid (cefdinir crystal B) which comprises the steps of:
preparing a cefdinir.trifluoroacetic acid salt;
neutralizing the cefdinir.trifluoroacetic acid salt by treatment with a base in water at a temperature between about 0° C. to 30° C.; and
isolating cefdinir crystal B by filtration.

17. A process for preparing the crystalline form of cefdinir of claim 1, comprising the steps of:
reacting crystals A of cefdinir in water with trifluoroacetic acid at about 35-40° C. to form cefdinir trifluoroacetic acid salt;
optionally isolating the cefdinir.trifluoroacetic acid salt;
neutralizing the cefdinir.trifluoroacetic acid salt by treatment with a base in water at a temperature between about 0° C. to 30° C.; and
isolating cefdinir crystal B by filtration.

18. A process for preparing the crystalline form of cefdinir of claim 1, comprising the steps of:
preparing a cefdinir.trifluoroacetic acid salt;
neutralizing the cefdinir.trifluoroacetic acid salt by treatment with a base in water at a temperature between about 0° C. to 30° C.; and
isolating cefdinir crystal B by filtration.

19. A process for preparing the crystalline form of cefdinir of claim 4, comprising the steps of:
reacting crystals A of cefdinir in water with trifluoroacetic acid at about 35-40° C. to form cefdinir trifluoroacetic acid salt;
optionally isolating the cefdinir.trifluoroacetic acid salt;
neutralizing the cefdinir.trifluoroacetic acid salt by treatment with a base in water at a temperature between about 0° C. to 30° C.; and
isolating cefdinir crystal B by filtration.

20. A process for preparing the crystalline form of cefdinir of claim 4, comprising the steps of:
preparing a cefdinir.trifluoroacetic acid salt;
neutralizing the cefdinir.trifluoroacetic acid salt by treatment with a base in water at a temperature between about 0° C. to 30° C.; and
isolating cefdinir crystal B by filtration.

21. A process for preparing the crystalline form of cefdinir of claim 8, comprising the steps of:
reacting crystals A of cefdinir in water with trifluoroacetic acid at about 35-40° C. to form cefdinir trifluoroacetic acid salt;
optionally isolating the cefdinir.trifluoroacetic acid salt;
neutralizing the cefdinir.trifluoroacetic acid salt by treatment with a base in water at a temperature between about 0° C. to 30° C.; and
isolating cefdinir crystal B by filtration.

22. A process for preparing the crystalline form of cefdinir of claim 8, comprising the steps of:
preparing a cefdinir.trifluoroacetic acid salt;
neutralizing the cefdinir.trifluoroacetic acid salt by treatment with a base in water at a temperature between about 0° C. to 30° C.; and
isolating cefdinir crystal B by filtration.
